(19) United States
(12) Patent Application Publication
(10) Pub. No.: US 2007/0173921 A1
(43) Pub. Date: Jul. 26, 2007

(54) FLARED STENTS AND APPARATUS AND METHODS FOR DELIVERING THEM

(76) Inventors: Mark H. Wholey, Pittsburgh, PA (US); Farhad A. Khosravl, Los Altos Hills, CA (US); James H. Dreher, Santa Monica, CA (US); Jeffrey A. Krolik, Campbell, CA (US)

Correspondence Address: Vista IP Law Group LLP, 2040 MAIN STREET, 9TH FLOOR, IRVINE, CA, 92614, US

(21) Appl. No.: 11/554,560

(22) Filed: Oct. 30, 2006

Related U.S. Application Data
(60) Provisional application No. 60/731,568, filed on Oct. 28, 2005.
(60) Provisional application No. 60/732,628, filed on Nov. 1, 2005.

(51) Int. Cl. A61F 2/06

(52) U.S. Cl. 623/1.13; 623/1.15; 623/1.31

(57) ABSTRACT

Apparatus and methods are provided for securing a stent-graft deployed within an aorta relative to a renal artery. A distal end of a delivery device may be introduced into the aorta, the distal end carrying a stent thereon. The distal end may be advanced through an opening in the stent-graft at least partially into the renal artery, and the stent may be expanded to anchor the stent-graft relative to the renal artery and/or secure the stent relative to the renal artery. An exemplary flaring stent is also disclosed.

This application claims benefit of provisional applications Ser. Nos. 60/731,568, filed Oct. 28, 2005, and 60/732,628, filed Nov. 1, 2005, the entire disclosures of which are expressly incorporated by reference herein.

### FIELD OF THE INVENTION

The present invention relates generally to endoluminal prostheses or “stents” or “stent-grafts”, and, more particularly, to flared stents, and to apparatus and methods for delivering such stents into an ostium of a blood vessel or other body lumen, e.g., to secure a stent-graft.

### BACKGROUND

Tubular endoprosthesis or “stents” have been suggested for dilating or otherwise treating stenoses, occlusions, and/or other lesions within a patient's vasculature or other body lumens. For example, a self-expanding stent may be maintained on a catheter in a contracted condition, e.g., by an overlying sheath or other constraint, and delivered into a target location, e.g., a stenosis within a blood vessel or other body lumen. When the stent is positioned at the target location, the constraint may be removed, whereupon the stent may automatically expand to dilate or otherwise line the vessel at the target location. Alternatively, a balloon-expandable stent may be carried on a catheter, e.g., crimped or otherwise secured over a balloon, in a contracted condition. When the stent is positioned at the target location, the balloon may be inflated to expand the stent and dilate the vessel.

Sometimes, a stenosis or other lesion may occur at an ostium or bifurcation, i.e., where a branch vessel extends from a main vessel or trunk. For example, such a lesion may form within a coronary artery immediately adjacent the aortic root. U.S. Pat. No. 5,749,890 to Shaknovich discloses a stent delivery assembly for placing a stent in an ostial lesion. U.S. Pat. No. 5,632,762 to Myler discloses a tapered balloon on a catheter for positioning a stent within an ostium. U.S. Pat. No. 5,607,444 to Lam discloses an expandable ostial stent including a tubular body and a deformable flaring portion. Published application US 2002/0077691 to Nachtigall discloses a delivery system that includes a sheath for holding a stent in a compressed state during delivery and a retainer that holds a deployable stop in an undeployed position while the delivery system is advanced to a desired location.

Accordingly, stents and apparatus and methods for delivering stents within an ostium would be useful.

### SUMMARY OF THE INVENTION

The present invention is directed to endoluminal prostheses or “stents,” and, more particularly, to flared stents, and to apparatus and methods for delivering such stents into an ostium of a blood vessel or other body lumen.

In accordance with one embodiment, a stent is provided that includes a tubular member including first and second ends defining a longitudinal axis therebetween and a plurality of cells disposed between the first and second ends, the tubular member being expandable from a contracted condition to an enlarged condition. The stent may include a first set of cells disposed at the first end, a second set of cells disposed adjacent the first set of cells, and a plurality of connectors coupling the first set of cells with the second set of cells such that radial expansion of the second set of cells towards the enlarged condition causes the first set of cells to flare radially outwardly.

In one embodiment, the first and second cells may include zigzag patterns including peaks and valleys, and the struts may connect respective peaks and valleys of the first and second sets of cells. In addition or alternatively, the struts coupling respective peaks of the first and second sets of cells may be longer than struts coupling respective valleys of the first and second sets of cells. In addition or alternatively, the zigzag patterns may include generally axial elements connecting the alternating peaks and valleys, and the axial elements in the first set of cells may be longer than the axial elements in the second set of cells.

In accordance with another embodiment, a stent is provided that includes a tubular member including first and second ends defining a longitudinal axis therebetween and a plurality of cells disposed between the first and second ends, the tubular member being expandable from a contracted condition to an enlarged condition. The stent may include a first flaring portion, and a second portion, the first flaring portion including a first set of cells disposed at the first end and a second set of cells disposed adjacent the first set of cells, the first set of cells defining an axial length that is longer than an axial length defined by the second set of cells.

The stent may also include a plurality of connectors coupling the first set of cells with the second set of cells such that radial expansion of the second set of cells towards the enlarged condition causes the first set of cells to flare radially outwardly.

In accordance with still another embodiment, an apparatus is provided for delivering a stent into an ostium. Generally, the apparatus may include an elongate member including a proximal end, a distal end sized for introduction into a body lumen, and an expandable member on the distal end; and a stent on the distal end. The stent may include a first flaring portion and a second portion, the second portion overlying the expandable member such that the expansion of the expandable member causes the second portion to expand radially.

The first flaring portion may be coupled to the second portion such that expansion of the second portion causes the first flaring portion to flare radially outwardly. In addition or alternatively, the first flaring portion may include a first band of cells adjacent a first end of the stent and a second band of cells between the first end and the second portion. The first and second bands of cells connected such that radial expansion of the second band of cells causes the first band of cells to flare radially outwardly.

The second set of cells may be coupled to the second portion such that radial expansion of the second portion causes the second set of cells to radially expand. Alternatively, the second set of cells may overly the expandable member such that expansion of the expandable member causes the second set of cells to expand radially.

In accordance with yet another embodiment, a method is provided for expanding a stent that includes providing a stent on an expandable member, the stent including first and second ends, a first set of cells at the first end, and a second set of cells adjacent the first set of cells; and expanding the expandable member to subject the second set of cells to a radially outward force that causes the second set of cells to expand radially outwardly, thereby causing the first set of cells to flare radially outwardly.

In one embodiment, the first set of cells may be coupled to the second set of cells such that the first set of cells flare radially outwardly when the second set of cells expand. In addition or alternatively, the first set of cells may flare radially outwardly away from the expandable member as the second set of cells expand such that the first set of cells move away from the expandable member.

In accordance with still another embodiment, a method is provided for delivering a stent within an ostium communicating between a main body lumen and a branch body lumen. The stent may be introduced into the main body lumen with the stent in a contracted condition, and positioned such that a first portion of the stent is disposed adjacent the ostium and a second portion of the stent is disposed within the branch body lumen. The second portion of the stent may be expanded within the branch body lumen, thereby causing the first portion of the stent to flare radially outwardly until the first portion engages the ostium.

In accordance with another embodiments, one or more flared stents may be delivered through fenestrations or other openings in a side wall of a stent-graft deployed within a main body lumen such that the stent(s) extend into branch body lumens communicating with the main body lumen. For example, for a AAA stent-graft deployed within the distal aorta, such stents may be delivered through openings in a stent-graft to obtain accurate alignment of the openings with the renal arteries or other branches. Such stents may also provide a smooth transition, with minimal flow disturbances between the aorta and the renal arteries. Such a stent may be delivered using a delivery catheter or other apparatus in conjunction with or after the AAA stent-graft is delivered. The stent may trap or otherwise secure material of the stent-graft between the stent and the vessel wall, e.g., using a flaring portion of the stent.

In accordance with one embodiment, an apparatus or system is provided for treating an aneurysm within a main body lumen that communicates with a branch body lumen. Generally, the apparatus includes a stent-graft and a flaring stent. The stent-graft may include a tubular body for implantation within a main body lumen across an aneurysm, the tubular body including at least one opening therethrough that may be aligned with a branch body lumen when the stent-graft is implanted across an aneurysm. The stent may include a first portion and a second portion, the second portion being receivable through the opening in the tubular body, the first portion being expandable to a flared condition for engaging the tubular body around the opening, e.g., for securing the stent-graft relative to the stent and/or to provide a substantially smooth transition with a branch body lumen within which the second portion is expanded.

In accordance with another embodiment, a method is provided for securing a stent-graft deployed within a main body lumen relative to a branch body lumen communicating with the main body lumen. A distal end of a delivery device may be introduced into the main body lumen, the distal end carrying a stent thereon. The distal end may be advanced through an opening in the stent-graft at least partially into the branch body lumen. The stent may be expanded to anchor the stent-graft relative to the branch body lumen and/or to provide a substantially smooth transition between the stent-graft and the branch body lumen.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a top view of a cell pattern for a stent having a flaring portion on one end.

FIG. 2 is a detail of the cell pattern of FIG. 1.

FIGS. 3A-3C are side views of a stent, showing a flaring portion of the stent flaring as the stent is expanded.

FIGS. 3D-3F are perspective views of the stent of FIGS. 3A-3C, respectively.

FIG. 4 is a perspective view of an apparatus for delivering a stent, including a guide catheter and a delivery catheter.

FIGS. 5A-5D are cross-sectional views of a patient's body, showing a method for implanting a stent, such as that shown in FIGS. 3A-3F, within an ostium of a body lumen.

FIG. 6 is a cross-sectional view of a patient's abdomen, showing an aneurysm within the patient's aorta immediately below the renal arteries.

FIG. 7 shows a stent-graft delivered across the aneurysm of FIG. 6, the stent-graft including openings aligned with the renal arteries.

FIG. 8 shows a guidewire, guide catheter, and delivery catheter introduced into the stent-graft of FIG. 7 for delivering a stent into one of the openings in the stent-graft.

FIGS. 9A-9C show a method for flaring and anchoring the stent of FIG. 8 in the ostium of the renal artery using the delivery device of FIG. 8.

FIG. 10 is a detail of the method of FIGS. 9A-9C, showing distal pressure being applied to anchor the stent to the vessel wall at the ostium, thereby securing the stent-graft between the stent and the vessel wall.

FIGS. 11A and 11B are details of a branch vessel adjacent an opening in a stent-graft. FIG. 11A shows a stent aligning and anchoring the opening in the stent-graft with the branch vessel, while FIG. 11B shows the risk of misalignment or migration of the stent-graft relative to the branch vessel without the stent.

### DETAILED DESCRIPTION OF THE PREFERRED EMBODIMENTS

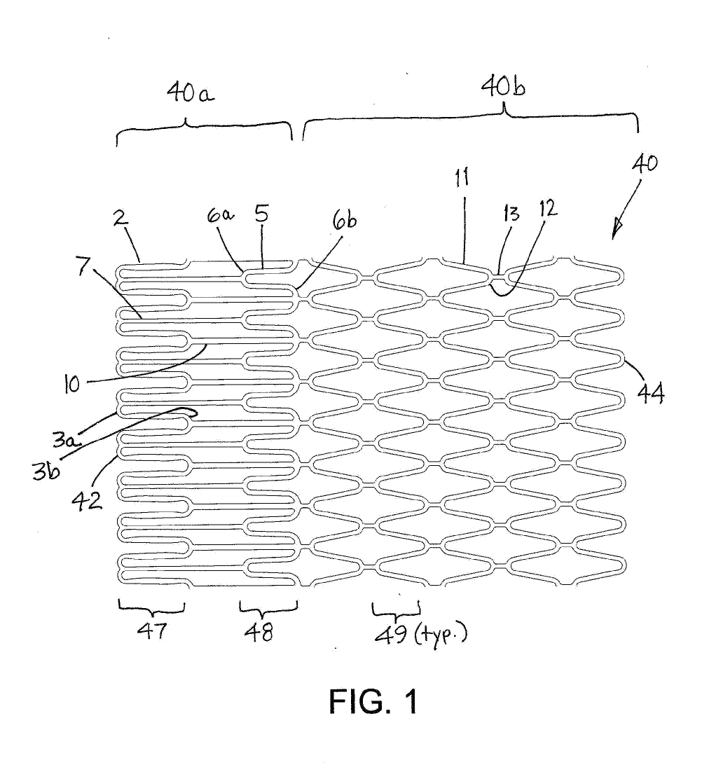

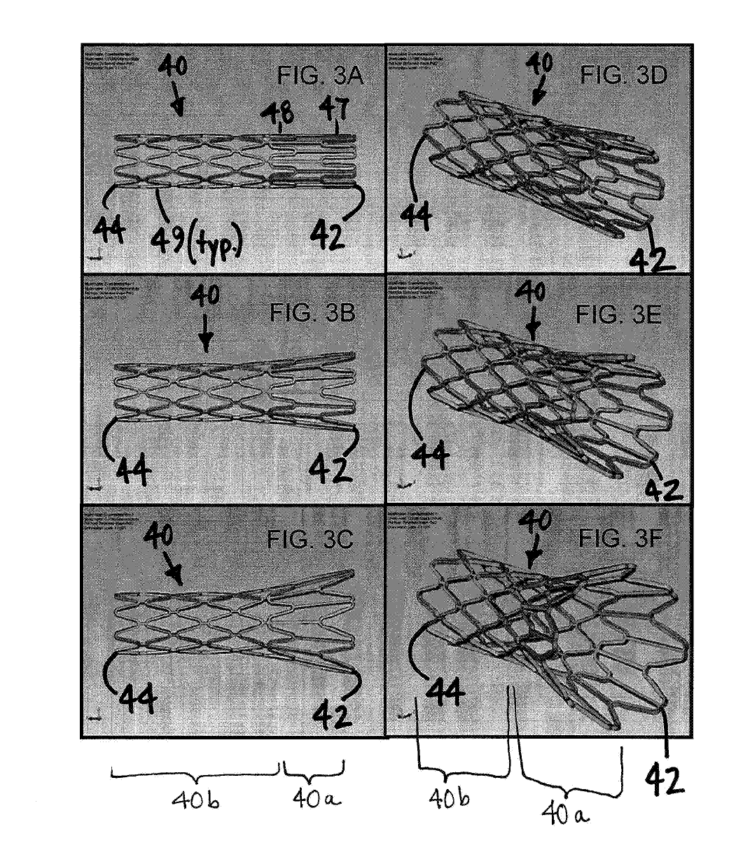

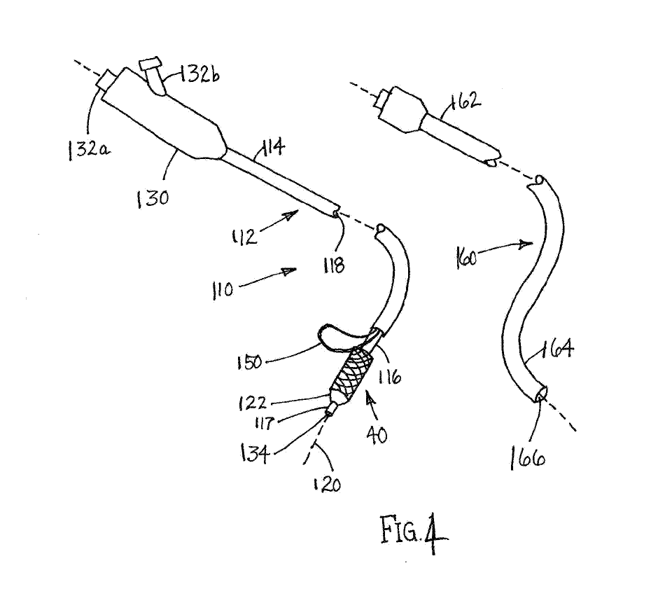

Turning to the drawings, FIGS. 1-3F show an exemplary embodiment of a stent 40 that includes a generally cylindrical tubular member including a proximal or first end 42 and a distal or second end 44 defining a longitudinal axis 46 therebetween. The stent 40 is generally radially expandable from a contracted or delivery condition (FIGS. 3A, 3D) to an enlarged or deployed condition (FIGS. 3C, 3F).

The stent 40 includes a plurality of annular bands of cells 47-49 disposed between the proximal and distal ends 42, 44. The bands of cells 47, 48 may generally define a first or flaring portion 40a of the stent 40, and the bands of cells 49 may define a second or main portion 40b of the stent 40. Each band of cells 47-49 may be defined by a plurality of struts or other elements extending axially along and/or circumferentially around the stent 40, e.g., in a zigzag or serpentine pattern, thereby defining an open-cell structure. Adjacent bands of cells may be connected to one another, e.g., directly or via links or other elements.

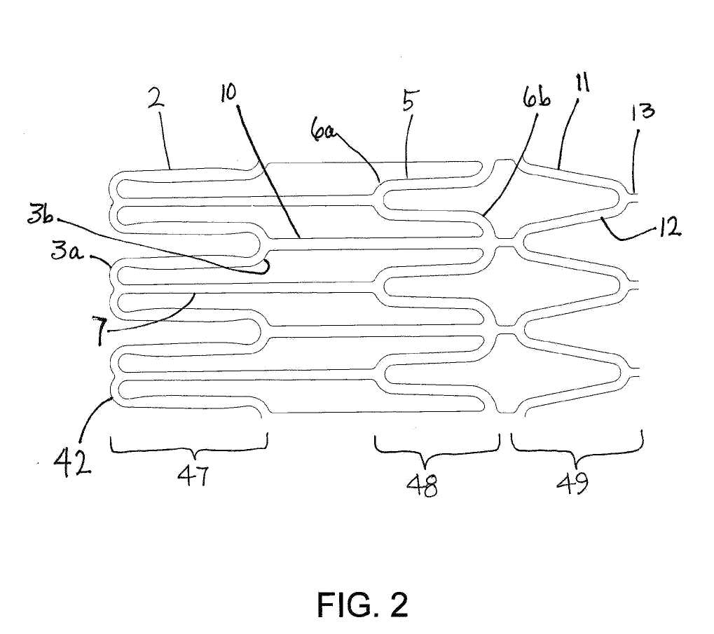

For example, as shown in FIGS. 1 and 3A, the stent 40 includes a first band of cells 47 at the first end 42 that includes a zigzag or serpentine pattern defined by a plurality of axial elements 2 connected alternately by curved elements 3 extending about the circumference of the stent 40. The axial elements 2 may be substantially straight, e.g., extending substantially parallel to the longitudinal axis in the contracted condition, as shown in FIG. 1. Alternatively, the axial elements 2 may include more complicated geometry, e.g., including one or more curves or bends, thereby including both an axial component and a circumferential component (not shown). Generally, with additional reference to FIG. 2, the first band of cells 47 includes a first axial length 16 substantially parallel to the longitudinal axis, which may be defined at least partially by a length of the axial elements 2, e.g., depending upon whether the axial elements 2 extend substantially parallel to the longitudinal axis or extend at an angle relative to the longitudinal axis (i.e., diagonally or circumferentially).

Similarly, the stent 40 includes a second band of cells 48 adjacent the first band of cells 47 that includes a zigzag or serpentine pattern defined by axial elements 5 connected alternately by curved elements 6 extending about the circumference of the stent 40. The axial elements 5 may be substantially straight, e.g., extending substantially parallel to the longitudinal axis in the contracted condition, as shown in FIG. 1. Alternatively, the axial elements 5 may include more complicated geometry, e.g., including one or more curves or bends, thereby including both an axial component and a circumferential component (not shown). Generally, with additional reference to FIG. 2, the second band of cells 48 includes a first axial length 15 substantially parallel to the longitudinal axis, which may be defined at least partially by a length of the axial elements 5, e.g., depending upon whether the axial elements 5 extend substantially parallel to the longitudinal axis or extend at an angle relative to the longitudinal axis (i.e., diagonally or circumferentially).

As shown, the first and second bands of cells 47, 48 may be substantially in-phase with one another around the circumference of the stent 40. Stated differently, the peaks and valleys of the first and second bands of cells 47, 48 may be aligned substantially axially relative to one another. For example, the curved elements 3a closer to the first end 42 (the peaks of the first band of cells 47) may be disposed generally axially relative to the curved elements 6a closer to the first end 42 (the peaks of the second band of cells 48). Similarly, the curved elements 3b further from the first end 42 (the valleys of the first band of cells 47) may be disposed generally axially from the curved elements 6b further from the first end 42 (the valleys of the second band of cells 48). Thus, the zigzag patterns of the first and second bands of cells 47, 48 may include the same number of axial elements 2, 5 and curved elements 3, 6. It will be appreciated that the terms peaks and valleys have been assigned to the zigzag patterns as a convenience to facilitate the relationship of the components of the stent 40, and no other special meaning is intended.

In addition, the second band of cells 48 are connected to the first band of cells 47 by one or more struts or other connectors 7, 10. Generally, the struts 7, 10 extend substantially axially between adjacent peaks and valleys of the zigzag patterns of the first and second bands of cells 47, 48. For example, a strut 7 may extend between each adjacent peak of the first and second bands of cells 47, 48, i.e., between the curved elements 3a, 6a closer to the first end 42. Similarly, struts 10 may extend between each adjacent valley of the first and second bands of cells 47, 48, i.e., between the curved elements 3b, 6b further away from the first end 42. It will be appreciated that some of the struts 7, 10 may be eliminated if desired, e.g., every one, two, three, or more struts around the circumference of the stent 40, depending upon the desired rigidity and/or flaring desired.

With additional reference to FIG. 2, the first axial length 16 may be substantially longer than the second axial length 15, e.g., by providing axial elements 2 of the first band of cells 47 that are substantially longer than axial elements 5 of the second band of cells 48. Consequently, the peak struts 7 may be relatively longer than the valley struts 10 in order to connect the first and second bands of cells 47, 48. As described further below, these differences in length may cause the first end 42, i.e., at least the first band of cells 47 to buckle and/or flare radially outwardly, e.g., in response to radial expansion of the second band of cells 48.

In addition, the stent 40 may include a plurality of additional bands of cells 49 defining the second portion 40b of the stent 40. Each of the additional bands of cells 49 may include axial elements 11 connected alternately to curved elements 12, thereby defining a zigzag or serpentine and third axial length. For example, each of the bands of cells 49 may have similar amplitudes and/or periods as the first and second bands of cells 47, 48. As shown, adjacent bands of cells 49 may be offset one hundred eighty degrees (180°) from one another such that pairs of curved elements 12 are disposed axially adjacent one another.

Optionally, adjacent bands of cells 49 defining the second portion of the stent 40 may be connected via links or connectors 13, as shown. For example, the links 13 may be axial struts extending between adjacent pairs of curved elements 12. Alternatively, the links may define at least a portion of a generally sinusoidal wave or other curvilinear shape (not shown), such as those disclosed in application Ser. No. 11/466,439, filed Aug. 22, 2006, the entire disclosure of which is expressly incorporated by reference herein. In a further alternative adjacent bands of cells 49 may be connected directly, e.g., by adjacent curved elements 12 (also not shown). Optionally, the links 13 may be relatively narrow and/or thin compared to the curved elements 12, e.g., to facilitate bending or conformability of the second portion 143 of the stent 140., or directly (not shown).

Although each of the bands of cells 49 in the second portion of the stent 40 are shown having similar configurations and axial lengths, it will be appreciated that the dimensions and configurations may be varied between the second band of cells 48 and the second end 44 of the stent 40, if desired. Thus, the second portion 40b of the stent 40 between the second band of cells 48 and the second end 44 of the stent may have a substantially homogenous cell structure or non-uniform cell and/or band configurations, e.g., as described further below. In addition, any number of annular bands 49 may be provided, e.g., such that the second portion 43 has a predetermined length corresponding to a length of a lesion being dilated or otherwise treated using the stent 40, e.g., between about three and twenty millimeters (3-20 mm).

Alternatively, the second (e.g., non-flaring) portion 40b of the stent 40 may include other configurations. For example, the second portion 40b may include cells that extend circumferentially, axially, and/or helically along the second portion 40b. The cells may be formed from slotted tubes, rolled sheets, and/or other materials, as described elsewhere herein. Alternatively, the second portion 40b may be formed from one or more wire structures, e.g., one or more helical wires extending from the first (e.g., flaring) portion to the second end 44, a braid of multiple wires, and the like (not shown). Thus, in some embodiments, the second portion 40b may be formed from any known stent structure or configuration, while the first portion 40a has the flared configuration described herein.

The stent 40 may be formed from a variety of materials that may be plastically deformed to allow expansion of the stent 40. For example, the stent 40 may be formed from metal, such as stainless steel, tantalum, MP35N, Niobium, Nitinol, and L605, plastic, or composite materials. In particular, the materials of the stent 40 may be plastically deformed under the pressures experienced when the stent 40 is expanded, e.g., such that the first and/or second portions 40a, 40b of the stent 40 are deformed beyond their elastic limit. Thus, when the stent 40 is deployed, the stent 40 may maintain its enlarged condition, e.g., that shown in FIGS. 3C and 3F and described further below. Stated differently, the stent 40 material may resist collapsing back towards its reduced configuration after deployment, e.g., if the tissue surrounding the body lumen attempts to constrict or otherwise return to its occluded shape.

Alternatively, at least a portion of the stent 40 may be self-expanding. For example, one or both of the first and second portions 40a, 40b may be biased to expand at least partially outwardly yet may be constrained on a delivery device in a contracted condition to facilitate delivery. In this alternative, the stent 40 may be formed from Nitinol or other shape memory or superelastic materials. The material may have the enlarged (and flared condition) programmed into the material, e.g., using heat treatment and the like. The stent 40 may then be constrained in a contracted condition, and deployed at a delivery site, whereupon the stent 40 may resiliently expand to the enlarged and flared condition.

In one embodiment, the stent 40 may be formed from a tube of material having a solid wall initially. For example, portions of the tube may be removed, e.g., by laser cutting, etching, machining, and the like, to define the elements of the bands of cells and/or links. Alternatively, the stent 40 may be formed from a flat sheet and rolled into a tubular shape. Portions of the sheet may be removed and then the resulting cellular structure may be rolled and attached along its length, e.g., by welding, bonding, interlocking connectors (not shown), and the like.

Optionally, the resistance of the stent 40 to expansion may be varied along its length, e.g., along the length of the second portion 40b. This performance of the stent 40 may be based upon mechanical properties of the material, e.g., which may involve heat treating one or more portions of the stent 40 differently than other portions. In addition or alternatively, the structure of the stent 40 may be varied, e.g., by providing struts, fibers, or other components in different bands of cells 49 having different widths, thicknesses, geometry, and the like, e.g., as described in application Ser. No. 11/439,717, filed May 23, 2006, the entire disclosure of which is expressly incorporated by reference herein.

If desired, one or more portions of the stent 40 may include a membrane, film, or coating (not shown), e.g., to create a nonporous, partially porous, or porous surface between cells of the stent 40, as described in application Ser. No. 11/439,717, incorporated by reference above. Optionally, the membrane may carry therapeutic or other compounds or materials. In addition or alternatively, the stent 40 may carry one or more therapeutic or other compounds (not shown) that may enhance or otherwise facilitate treatment of a target location within a patient's body. For example, the stent 340 may carry compounds that prevent restenosis at the target location. Optionally, the stent 40 may include one or more radiopaque or other markers (not shown), e.g., to facilitate monitoring the stent during advancement, positioning, and/or expansion, as described in application Ser. No. 11/466,439, incorporated by reference above.

Turning to FIG. 4, the stent 40 may be delivered to a target location within a patient's body using a delivery apparatus, such as the apparatus 110. Generally, the apparatus 110 includes a catheter or other elongate tubular member 112 having a proximal end 114, a distal end 116, and one or more lumens 118 extending between the proximal and distal ends 114, 116, thereby defining a longitudinal axis 120 between the proximal and distal ends 114, 116.

One or more balloons or other expandable members 122 may be provided on the distal end 116 of the delivery catheter 112 for expanding and/or deploying the stent 140, as described further below. Optionally, the delivery catheter 112 may include one or more locator elements, such as locator loop 150 on the distal end 116, e.g., proximal or otherwise adjacent to the stent 40. Alternatively, the delivery catheter 112 may include multiple locator loops (not shown), an expandable locator element, e.g., a balloon (not shown) proximal to the stent 40 and balloon 122, and the like.

In addition, the apparatus 110 may include a guide catheter 160 including a proximal end 162, a distal end 164, and a lumen 166 extending therebetween. The distal end 164 may be sized and/or shaped to facilitate advancement into a patient's vasculature or other body lumen, as described further below. The lumen 166 may have sufficient size for receiving the distal end 116 of the delivery catheter 112 therethrough, e.g., with the locator loop 150 in a contracted condition. Optionally, the distal end 164 of the guide catheter 160 may be biased to a predetermined shape, e.g., a “J” shape, which may facilitate positioning the guide catheter 160 within or adjacent an ostium. Optionally, the apparatus 110 may include other components to provide a system or kit for delivering the stent 40, e.g., a sheath that may be advanced over and/or retracted from the distal end 116 of the delivery catheter 112, one or more syringes or other sources of inflation media and/or vacuum, tubing, and/or one or more guidewires (all not shown).

With continued reference to FIG. 4, the delivery catheter 112 may be formed from one or more tubular bodies, e.g., having variable flexibility along its length. For example, the distal end 116 may be substantially flexible to facilitate insertion through tortuous anatomy, e.g., terminating in a rounded, tapered, and/or other substantially atraumatic distal tip 117. The distal end 116 may be sized and/or shaped for introduction into a body lumen, e.g., having a diameter between about one and seven millimeters (1-7 mm), or less than 1.5 millimeters. The proximal end 114 may be substantially flexible or semi-rigid, e.g., having sufficient column strength to facilitate advancing the distal end 116 through a patient's vasculature by pushing on the proximal end 114. The delivery catheter 112 may be formed from plastic, metal, or composite materials, e.g., a plastic material having a wire, braid, or coil core, which may preventing kinking or buckling of the delivery catheter 112 during advancement.

As shown in FIG. 4, the delivery catheter 112 may include a handle 130 on the proximal end 114, e.g., to facilitate manipulating the delivery catheter 112. The handle 130 may include one or more side ports 132 communicating with respective lumens 118 within the delivery catheter 112. The proximal end 114 of the delivery catheter 112 may be attached to the handle 130, e.g., by bonding, cooperating connectors, interference fit, and the like. Optionally, if the apparatus 110 includes any actuatable components (not shown) on the distal end 116, the handle 130 may include one or more actuators (not shown), such as one or more slides, dials, buttons, and the like, for actuating or otherwise manipulating the components on the distal end 116 from the proximal end 114.

In the embodiment shown in FIG. 4, the delivery catheter 112 includes at least two lumens 118 extending between the proximal and distal ends 114, 116. For example, the delivery catheter 112 may include a guidewire or instrument lumen that extends from a port 132a in the handle 130 to an opening 134 in the distal tip 117. The instrument lumen may have sufficient size to allow a guidewire or other rail or instrument (not shown) to be inserted therethrough, e.g., to facilitate advancing the delivery catheter 112 over the rail, as explained further below. Optionally, the handle 130 may include one or more seals (not shown) within or adjacent the port 132a, e.g., e.g., a hemostatic seal that prevents fluid, e.g., blood, from flowing proximally out of the port 132a, yet allows one or more instruments to be inserted therethrough and into the instrument lumen.

In addition, the delivery catheter 112 may include an inflation lumen that extends from side port 132b in the handle 130 through the delivery catheter 112 to an opening (not shown) that communicates with an interior of balloon 122. The side port 132b may include one or more connectors, e.g., a luer lock connector (not shown), one or more seals (also not shown), and the like. A source of inflation media and/or vacuum, e.g., a syringe filled with saline (not shown), may be connected to the side port 132b, e.g., via tubing (also not shown), for expanding and/or collapsing the balloon 122.

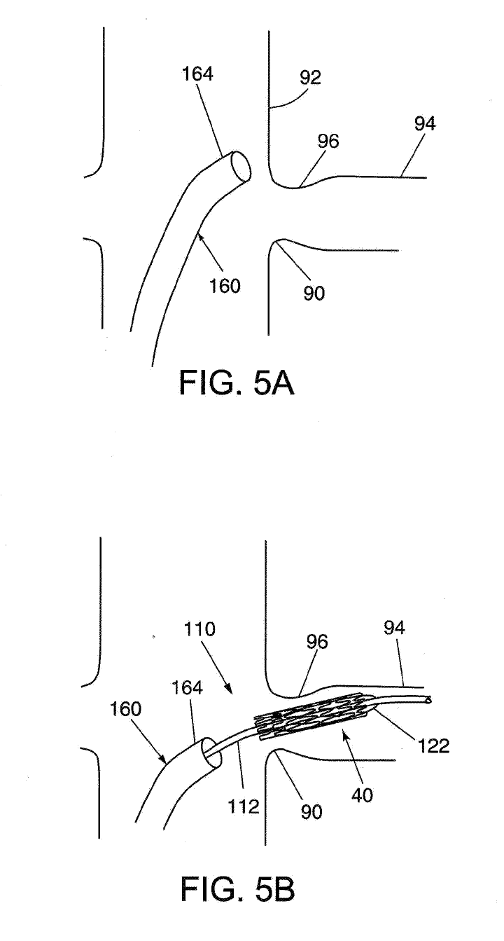

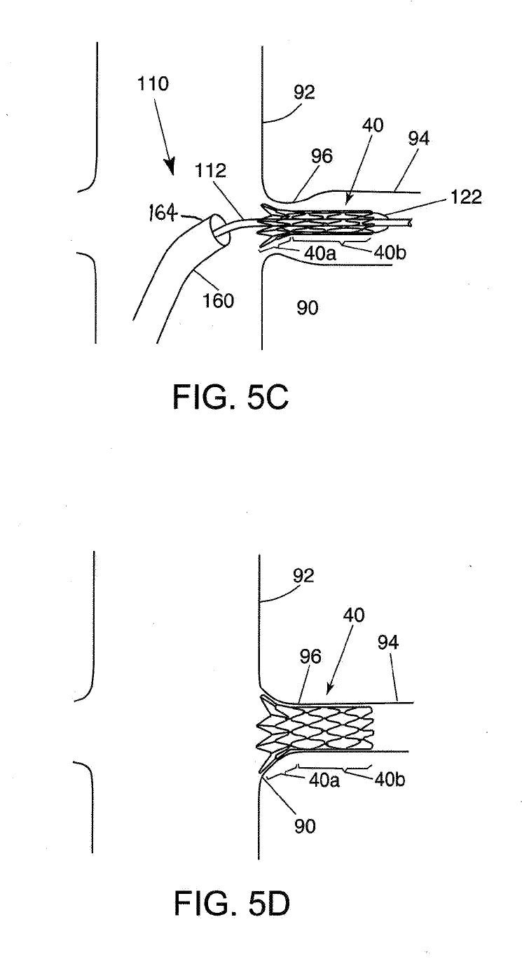

The balloon 122 may be bonded or otherwise secured to the distal end 116 of the delivery catheter 112. For example, ends of the balloon 122 may be attached to the distal end 116 using one or more of bonding with an adhesive, sonic welding, an annular collar or sleeve, and the like. The balloon 122 may be expandable from a contracted condition, as shown in FIGS. 4 and 5B, which may facilitate advancement through a patient's vasculature, to an enlarged condition for expanding or otherwise deploying the stent 140, as shown in FIG. 5C.

The balloon 122 may be formed from substantially inelastic material, e.g., PET, nylon, or PEBAX, such that the balloon 122 expands to a predetermined size in its enlarged condition once sufficient fluid is introduced into the interior of the balloon 122. Alternatively, the balloon 122 may be formed from substantially elastic material, e.g., silicone, polyurethane, or polyethylene, such that the balloon 122 may be expanded to a variety of sizes depending upon the volume and/or pressure of fluid within the interior. Additional information on the apparatus 110 or other delivery apparatus that may be used for delivering the stent 40 may be found in applications Ser. Nos. 11/419,997, filed May 23, 2006 and 11/537,569, filed Sep. 29, 2006, the entire disclosures of which are expressly incorporated by reference herein.

Returning to FIGS. 3A-3F, the stent 40 may be provided initially in the contracted condition shown in FIGS. 3A, 3D, e.g., having a diameter between about one half and two millimeters (0.5-2 mm). The stent 40 may be delivered endoluminally, e.g., using the apparatus 110, as described further below. The stent 40 may then be expanded to the enlarged condition shown in FIGS. 3C, 3F, e.g., using the balloon 122 or other expandable member (not shown).

In the enlarged condition, both of the first and second portions 40a, 40b of the stent 40 define a circumference or other cross-sectional dimension that is larger than in the contracted condition. More particularly, the first portion 40a of the stent 40 may be expanded to assume a flared shape, e.g., having an outer diameter between about four and fifteen millimeters (4-15 mm), while the second portion 40b of the stent 40 may be expanded to a generally uniform cylindrical shape, e.g., having a diameter between about two and seven millimeters (2-7 mm).

Turning to FIGS. 3C, 3F, the flared shape of the first band of cells 47 will now be described in more detail, i.e., after the stent 40 has been expanded to the enlarged condition. Because of the difference in lengths between the first and second bands of cells 47, 48, the first portion 40a of the stent 40 flares radially outwardly as it expands. This flaring may be created by the mismatch of the first and second axial lengths 47, 48 (see FIG. 2), i.e., because the first band of cells 47 are substantially longer than the second band of cells 48, and the mismatch in lengths of the struts 7, 10.

For example, as described above with reference to FIG. 4, the stent 40 may be disposed on balloon 122 or other expandable member (not shown), e.g., on the distal end 116 of delivery catheter 112. The balloon 122 may be inflated, thereby applying a radially outward force on the stent 40. This force causes the bands 47-49 to expand radially outwardly. As the stent 40 expands, the axial elements 5 of the second band of cells 48 may be deflected from a substantially axial orientation in the contracted condition towards a more transverse or circumferential orientation in the expanded condition, thereby shortening the axial length of the second band of cells 48.

Because of the struts 7, 10, the shortening of the second band of cells 48 causes a corresponding shortening in the axial length of the first band of cells 47. However, because of the differences in lengths between the axial segments 2, 5 and the struts 7, 10, this shortening subjects the struts 7, 10 and axial segments 2 to a buckling force. To relieve this buckling force, the struts 7, 10 will deflect radially outwardly, thereby flaring the first band of cells 47 to flare radially outwardly. This may cause the first band of cells 47 to separate away from the balloon 122 and/or distal end 116 of the delivery catheter 112. Thus, the first end 42 may have a diameter or other cross-sectional dimension that is substantially larger than the transition between the first and second bands of cells 47, 48 and/or than the second end 44.

Stated differently, the first band of cells 47 may be flared simply because of the mechanical interaction of the first band of cells 47 with the second band of cells 48 and/or the other bands of cells 49. Because of this, it may be possible to flare the first portion 40a of the stent 40 without using a balloon or other expandable member to direct the first portion 40a radially outwardly towards the flared configuration. However, if desired, one or more flaring balloons (not shown) may be used to assist in the mechanical flaring of the first band of cells 47. For example, after flaring the first band of cells 47 by expanding the second band of cells 48 and/or the second portion 49, a proximal balloon (not shown) may be expanded to further expand and/or flare the first band of cells 47 (and, optionally, the second band of cells 48 as well if the proximal balloon at least partially underlies the second band of cells 48, e.g., over or under a proximal portion of the balloon 122).

Turning to FIGS. 5A-5D, to deliver the stent 40, the apparatus 110 or other apparatus, such as those disclosed in the applications identified above, may be provided. With additional reference to FIG. 4, the stent 40 may be mounted around the distal end 116 of the catheter 112, e.g., surrounding the balloon 122. In one embodiment, the entire stent 40 overlies the balloon 122, e.g., such that proximal and distal ends of the balloon 122 extend beyond the ends 42, 44 of the stent 40. Alternatively, only the second portion 40b of the stent 40 may overly the balloon 122 or the second portion 40b and the second band of cells 48 may overly the balloon 122.

Optionally, the apparatus 110 may include a sheath or other cover (not shown) that may surround or otherwise cover the stent 40. The sheath may be removable from over the proximal or distal portions of the stent 40 or the entire stent 40 to expose the stent 40 before deployment. Alternatively, if the stent 40 is self-expanding, the balloon 122 may be eliminated and/or the sheath may be used to constrain the stent 40 in the contracted condition until time of deployment.

The apparatus 110 may be used to deliver the stent 40 into an ostium 90, e.g., an opening in a wall of a first or main body lumen 92 that communicates with a second or branch body lumen 94. In an exemplary embodiment, the main body lumen 92 may be the aortic root and the branch body lumen 94 may be a coronary artery. Alternatively, the main body lumen 92 may be the distal aorta or other peripheral vessel, and the branch body lumen may be a renal artery or other peripheral branch. It will be appreciated that the apparatus and methods described herein may be applicable to a variety of bifurcations or branch body lumens that extend transversely, e.g., laterally or substantially perpendicular, from a main body lumen, e.g., within a patient's vasculature or other systems.

Initially, as shown in FIG. 5A, guide catheter 160 may be advanced into the main body lumen 92, e.g., until a distal end 164 of the guide catheter 160 is disposed adjacent or proximal to the ostium 90. For example, the guide catheter 160 may be introduced into the patient's body from a percutaneous puncture or other entry site (not shown), through the patient's vasculature and into the main body lumen 92 using known methods.

Optionally, a guidewire or other rail 98 may be introduced from the main body lumen 92 through the ostium 90 into the branch 94, e.g., via the guide catheter 160. For example, the guide catheter 160 may be advanced or otherwise manipulated until the distal end 164 is engaged in the ostium 90, and the guidewire 98 may be advanced through the guide catheter 160 and passed through the lesion 96. Alternatively, the guidewire 98 may be introduced before or independent of the guide catheter 160.

Turning to FIG. 5B, with the stent 40 in the contracted condition, the distal end 116 of the delivery catheter 112 may be advanced over the guidewire 98 and/or through the guide catheter 160 from the entry site into the main body lumen 92. The delivery catheter 112 may be positioned to place the stent 40 at least partially within the ostium, e.g., such that the first portion 40a is disposed within or adjacent the ostium 90 and the second portion 40b is disposed within the branch 94. For example, if the delivery catheter 112 includes locator loop 150 (or other locator element(s), not shown), the locator loop 150 may be deployed within the main body lumen 92. The delivery catheter 112 may be advanced into the branch 94 until the locator loop 150 contacts the ostium 90 and/or the wall of the main body lumen 92, thereby providing tactile feedback to the user.

In addition or alternatively, fluoroscopy or other external imaging may be used to facilitate positioning the delivery catheter 112, and consequently, the stent 40 relative to the ostium 90. Thus, the stent 40 may be positioned such that the first portion 40a is disposed adjacent and/or within the ostium 90 and the second portion 40b extends into the branch 94.

As shown in FIG. 5C, once the stent 40 is properly positioned, the balloon 122 on the delivery catheter 112 may be expanded to expand the stent 40, thereby expanding the second band of cells 48. For example, the balloon 122 may apply a radially outward force against one or more portions of the stent 40, thereby causing the second band of cells 48 to expand radially. As the second band of cells 48 expand, the struts 7, 10 may at least partially buckle, thereby expanding and flaring the first band of cells 47 and the first end 42 of the stent 40 is expanded, e.g., until the first end 42 substantially engages the ostium 90 and/or the second end 44 engages the wall of the branch 94.

For example, the balloon 122 may apply a radially outward force against the second portion 40b of the stent 40, thereby expanding the second portion 40b. As the second portion 40b expands, the second band of cells 48 may be forced radially outwardly because the second band of cells 48 is coupled to the second portion 40b, e.g., by struts. Alternatively, the balloon 122 may apply a radially outward force upon the first and/or second band of cells 47, 48. As the first band of cells 47 begins to flare radially outwardly, the first band of cells may move away from the balloon 122 as they flare radially outwardly. For example, the first set of cells 47 may flare radially outwardly away from the balloon 122 as the second set of cells 48 expand such that the balloon 122 does not apply a direct radially outward force on the first set of cells 47 (if it ever did).

Thus, the stent 40 may substantially simultaneously dilate and/or secure the second portion 40b of the stent 40 within the branch 94 (and/or the lesion between the branch 94 and ostium 90) as the first portion 40b expands and/or flares radially outwardly to secure and/or dilate the ostium 90.

Turning to FIG. 5D, once the stent 40 is fully expanded and/or deployed, the balloon 122 may then be deflated, and the apparatus 110 removed, leaving the stent 40 within the ostium 90 and branch 94. For example, the guide catheter 160 may be directed against the ostium 90 to prevent withdrawal of the stent 40, and then the delivery catheter 112 and/or guidewire 98 may be withdrawn into the guide catheter 160. The guide catheter 160 may then be withdrawn, leaving the stent 40 within the ostium 90.

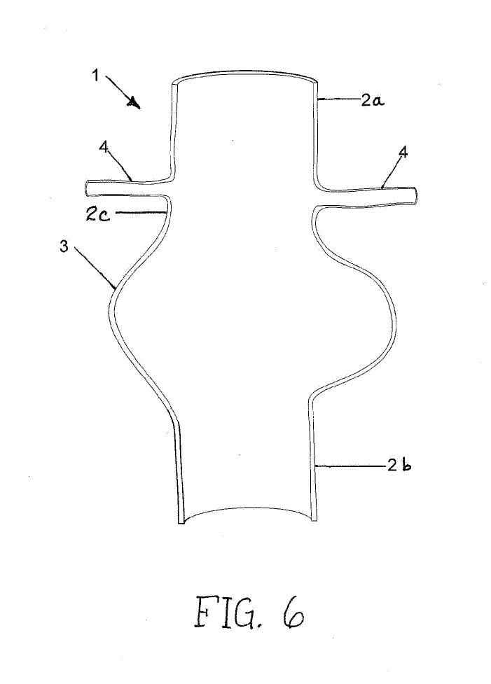

Turning to FIG. 6, an aortic abdominal aneurysm (“AAA”) generally refers a condition in which a wall of a blood vessel, i.e., the distal aorta, weakens. Because of the weakened wall, the aorta may expand under normal blood pressure, creating a large cavity in which blood may pool. Ultimately, the wall may weaken until it ruptures, which may cause serious immediate trauma or even death. Such aneurysms commonly occur immediately below the renal arteries and may extend to the aorto-iliac bifurcation or even into one or both iliac arteries.

Tubular grafts or stent-grafts may be used to treat AAA conditions. Tubular grafts may be implanted surgically, i.e., using open surgical procedures. Stent-grafts may be delivered endoluminally, e.g., using one or more catheters introduced from a percutaneous entry into the aneurysm. For example, a catheter may be introduced into a femoral artery below the aneurysm and advanced retrograde into the aorta, where the stent-graft may be deployed and secured across the aneurysm.

Generally, the stent-graft is secured at its proximal end (which herein refers to the end closest to the entry site) and its distal end (which herein refers to the end furthest from the entry site) within healthy portions of the vessel on either side of the aneurysm. Depending upon the location of the aneurysm, the stent-graft may have different configurations. For example, the stent-graft may have a tubular configuration, e.g., if the aneurysm is located above the aorto-iliac bifurcation. Alternatively, if the aneurysm extends into the iliac arteries, the stent-graft may have a “Y” or “pair of pants” configuration, e.g., with legs of the stent-graft extending into the iliac arteries.

It is common to attach the distal (or upper) end of the stent-graft immediately below the renal arteries. However, if the aneurysm extends upwards towards the renal arteries, it may be difficult to effectively seal and/or anchor the proximal end of the stent graft below the renal arteries. In such circumstances, it may be desirable to attach the distal end of the stent-graft above the renal arteries. In order to allow continued blood flow into the renal arteries, the stent-graft may include holes or fenestrations in its side wall that may communicate with the renal arteries.

With continued reference to FIG. 6, a section view of a segment of an abdominal aorta 1 is shown having healthy or normal diameter sections 2, an aneurysm 3 with a diameter larger than the healthy sections 2, and renal arteries 4 extending from the aorta 1. The section 2c of the aorta 1 inferior to the renal arteries 4 (between the renal arteries 4 and the aneurysm 3) may have a healthy or normal diameter, but may have insufficient length to accommodate proper placement of a stent-graft (not shown). Thus, attempting to secure one end of a stent-graft below the renal arteries 4 may result in incomplete treatment of the aneurysm 3, i.e., insufficient sealing of the aneurysm 3 from the aorta 1 and/or insufficient anchoring of the stent-graft. The healthy section 2a of the aorta 1 superior to (above) the renal arteries 4 has a longer vessel length of constant, normal diameter, and may provide a better candidate location for placing a distal end of a stent-graft.

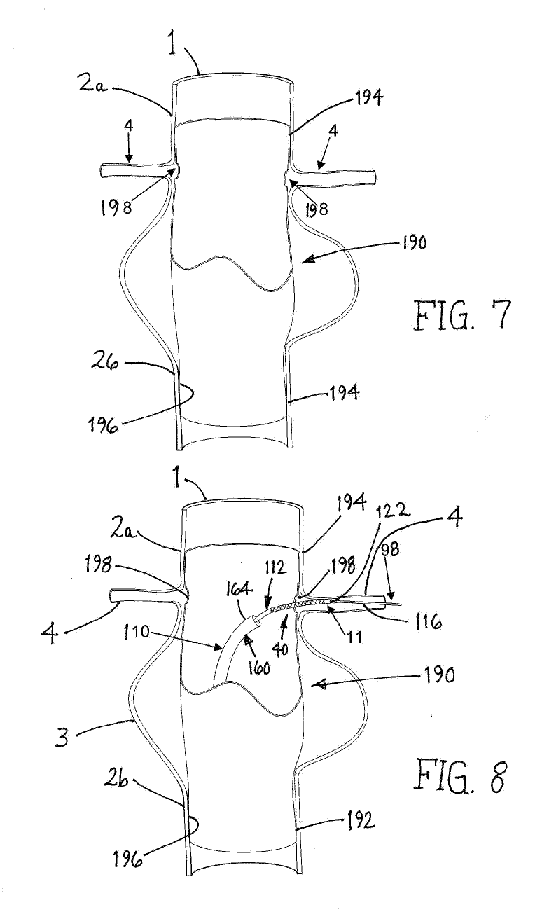

Turning to FIG. 7, a stent-graft 190 is shown (partially cut-away for clarity) that has been deployed across the aneurysm 3 of FIG. 6. Generally, the stent-graft 190 may include a tubular body including a proximal end 192, a distal end 194, and a lumen 196 extending between the proximal and distal ends 192, 194. The stent-graft 190 may have sufficient length to straddle a target aneurysm, i.e., such that the proximal and distal ends 192, 194 extend beyond the aneurysm 3 into healthy portions 2a, 2b of the aorta 1, as shown in FIG. 7.

The stent-graft 190 may be any known stent-graft, e.g., including a tubular structure, e.g., a tubular graft portion of Dacron or other fabric, that extends between the proximal and distal ends 192, 194. The fabric of the graft portion may have a desired porosity, e.g., may be substantially nonporous to blood or other fluids flowing within the target vessel, for isolating the aneurysm 3 from substantial fluid flow and/or pressure. Optionally, the stent-graft 190 may include an anchoring structure (not shown) on one or both of the proximal and distal ends 192, 194. For example, the stent-graft 190 may include a self-expanding stent or a balloon-expandable stent (not shown) on the proximal and/or distal ends 192, 194, which may be expanded to engage the wall of the aorta 1 to substantially secure the stent-graft 190 therein. Exemplary embodiments of stent-grafts that may be used are disclosed in U.S. Pat. Nos. 5,078,726, 5,151,105, 6,017,307, and 6,325,820.

The stent-graft 190 may include one or more fenestrations, holes, or other openings 198, e.g., in the tubular fabric or graft portion, which may be roughly aligned with corresponding branches extending from the aorta, e.g., two opposite openings 198 that may be aligned with the renal arteries 4. The openings 198 may formed simply by removing portions of the stent-graft 190, e.g., by cutting, boring, or otherwise removing circular portions of the graft portion at desired locations. If the graft portion is formed from fabric or other woven material, the edges may be sealed, e.g., fused, bonded with adhesive, stitched, and the like, e.g., to prevent subsequent fraying or deterioration of the stent-graft 190 around the openings 198. As shown in FIG. 7, although the openings 198 may be generally circular or otherwise shaped similar to the renal arteries 4, the openings 198 may not provide a smooth transition into the renal arteries 4. In fact, the openings 198 may provide abrupt transitions, e.g., if the edges of the openings 198 are sealed, which may cause significant flow disturbances resulting in additional vascular disease.

With continued reference to FIG. 7, the stent-graft 190 may be delivered endoluminally into the aorta or other main vessel or body lumen 1 having an aneurysm 3. For example, the stent-graft 190 may be provided initially in a contracted condition (not shown) and introduced into the patient's body using a catheter or other delivery apparatus (also not shown), e.g., from an entry site, such as a percutaneous entry site into a femoral artery, i.e., below the aorta 1. The catheter may be advanced into the aorta 1 and positioned such that the distal end 194 is disposed above the branch vessels 4, e.g., within the healthy upper portion 2a, and the proximal end 192 is disposed below the aneurysm 3, e.g., within the healthy lower portion 2b of the aorta 1.

The stent-graft 190 may then be deployed from the catheter, e.g., by removing an overlying sheath (not shown), using one or more balloons (also not shown) to expand the stent-graft 190, and the like. For example, stents coupled to the proximal and distal ends 192, 194 may be expanded within the aorta 1 to expand and/or secure the stent-graft 190 within the aorta 1. The stents may be self-expanding such that, upon exposure within the aorta 1, the stents resiliently expand to contact the wall of the aorta 1. In addition or alternatively, the stents may be expanded using one or more balloons to plastically deform the stents until the contact the wall of the aorta 1. The balloon(s) may also be used to unfold or otherwise expand the graft portion of the stent-graft 190 from the contracted condition towards the enlarged condition shown in FIG. 7.

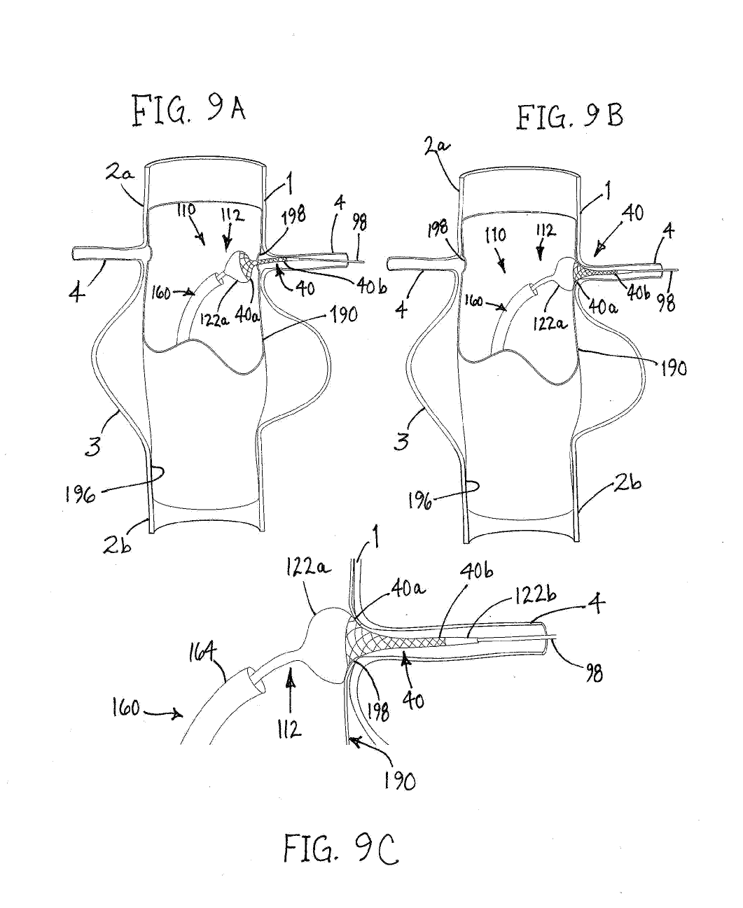

Turning to FIGS. 8-9C, an apparatus 110 and method are shown for delivering a flaring stent 40 into the openings 198. The stent 40 may provide a substantially smooth transition between the stent-graft 190 and the renal artery 4 and/or may anchor or enhance securing the stent-graft 190, as explained further below. The stent 40 may include the embodiments described above or in any of the references cited or incorporated by reference herein.

Generally, the apparatus 110 includes a delivery catheter or other elongate tubular member 112 having a proximal end (not shown), a distal end 116, and one or more lumens (also not shown) extending between the proximal end and the distal end 116, thereby defining a longitudinal axis therebetween. One or more balloons or other expandable members 122 may be provided on the distal end 116 of the delivery catheter 112 for expanding and/or deploying the stent 40, as described further below. Optionally, the delivery catheter 112 may include one or more locator elements (not shown) on the distal end 116, e.g., proximal or otherwise adjacent to the stent 40. Exemplary locator elements and apparatus and methods for using them may be found in US applications Ser. Nos. 11/419,997, filed May 23, 2006 and 11/537,569, filed Sep. 29, 2006, incorporated by reference above.

In addition, the apparatus 110 may include a guide catheter 160 including a proximal end (not shown), a distal end 164, and a lumen (not shown) extending therebetween. The distal end 164 may be sized and/or shaped to facilitate advancement into a patient's vasculature or other body lumen, as described further below. The lumen may have sufficient size for receiving the distal end 116 of the delivery catheter 112 therethrough. Optionally, the distal end 164 of the guide catheter 160 may be biased to a predetermined shape, e.g., a “J” shape, which may facilitate positioning the guide catheter 160 within or adjacent an ostium. Optionally, the apparatus 110 may include other components to provide a system or kit for delivering the stent 40, e.g., a sheath that may be advanced over and/or retracted from the distal end 116 of the delivery catheter 112, one or more syringes or other sources of inflation media and/or vacuum, tubing, and/or one or more guidewires (all not shown).

With continued reference to FIG. 8, the delivery catheter 112 may be formed from one or more tubular bodies, e.g., having variable flexibility along its length. For example, the distal end 116 may be substantially flexible to facilitate insertion through tortuous anatomy, e.g., terminating in a rounded, tapered, and/or other substantially atraumatic distal tip. The distal end 116 may be sized and/or shaped for introduction into a body lumen, e.g., having a diameter between about one and seven millimeters (1-7 mm), or less than 1.5 millimeters. The proximal end may be substantially flexible or semi-rigid, e.g., having sufficient column strength to facilitate advancing the distal end 116 through a patient's vasculature by pushing on the proximal end 114. The delivery catheter 112 may be formed from plastic, metal, or composite materials, e.g., a plastic material having a wire, braid, or coil core, which may preventing kinking or buckling of the delivery catheter 112 during advancement.

The delivery catheter 112 may include a handle (not shown) on the proximal end, e.g., to facilitate manipulating the delivery catheter 112. The handle may include one or more ports (also not shown) communicating with respective lumens within the delivery catheter 112. In the embodiment shown in FIG. 8, the delivery catheter 112 includes at least three lumens extending between the proximal end and the distal end 116. For example, the delivery catheter 112 may include a guidewire or instrument lumen that extends from a port in the handle to an opening in the distal end 11 6. The instrument lumen may have sufficient size to allow a guidewire or other rail or instrument (not shown) to be inserted therethrough, e.g., to facilitate advancing the delivery catheter 112 over the rail, as explained further below. In addition, the delivery catheter 112 may include inflation lumens that extend from the handle through the delivery catheter 112 to respective openings (not shown) that communicate with an interior of respective balloons 122. A source of inflation media and/or vacuum, e.g., a syringe filled with saline (not shown), may be connected to the handle, e.g., via tubing (also not shown), for expanding and/or collapsing the balloons 122.

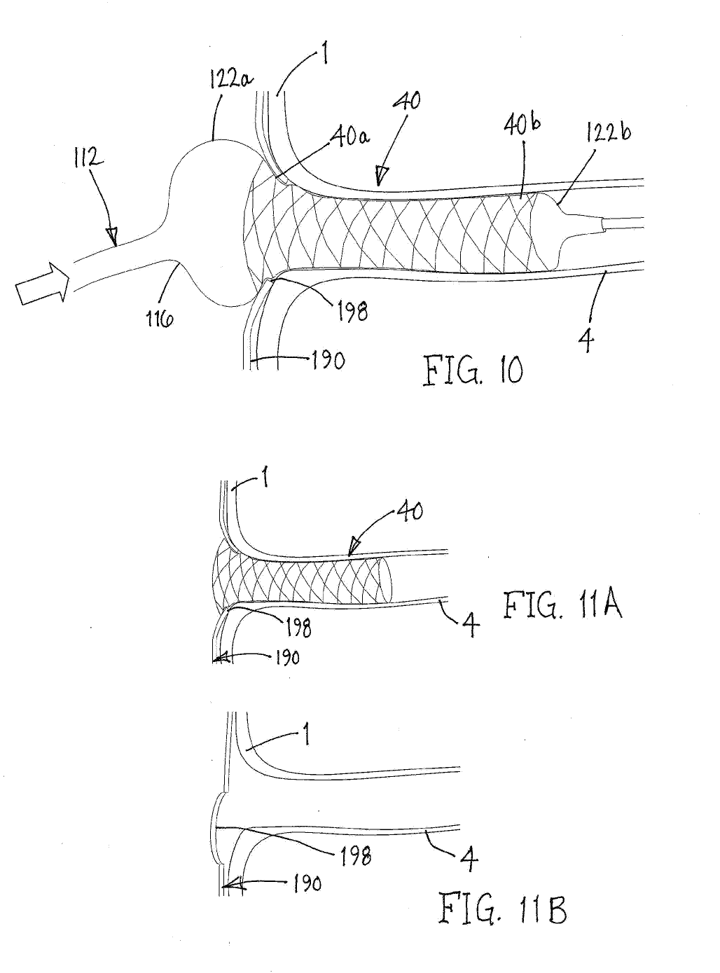

The balloons 122 may be bonded or otherwise secured to the distal end 116 of the delivery catheter 112. For example, ends of the balloons 122 may be attached to the distal end 116 using one or more of bonding with an adhesive, sonic welding, an annular collar or sleeve, and the like. The balloons 122 may be expandable from a contracted condition, as shown in FIG. 8, which may facilitate advancement through a patient's vasculature, to enlarged conditions for expanding or otherwise deploying the stent 40, as shown in FIGS. 9A-10.

The balloons 122 may be formed from substantially inelastic material, e.g., PET, nylon, or PEBAX, such that the balloons 122 expand to a predetermined size in its enlarged condition once sufficient fluid is introduced into the interior of the balloons 122. Alternatively, the balloons 122 may be formed from substantially elastic material, e.g., silicone, polyurethane, or polyethylene, such that the balloons 122 may be expanded to a variety of sizes depending upon the volume and/or pressure of fluid within the interior. Additional information on the apparatus 110 or other delivery apparatus that may be used for delivering the stent 40 may be found in applications Ser. Nos. 11/136,266, filed May 23, 2005, 11/419,997, filed May 23, 2006, 11/439,717, filed May 23, 2006, 11/466,439, filed Aug. 22, 2006, and 11/537,569, filed Sept. 29, 2006, the entire disclosures of which are expressly incorporated by reference herein.

Returning to FIG. 8, during use, after delivering the stent-graft 190, a guidewire 98 may be introduced into the patient's vasculature and advanced through the aorta 1 and stent-graft 190, and into one of the openings 198 and the corresponding renal artery 4. In addition or alternatively, a guide catheter 160 may be introduced into the vasculature and advanced into the aorta 1 and into the lumen 196 of the stent-graft 190 adjacent the opening 198, e.g., over the guidewire 98 or independent of the guidewire 98. The delivery catheter 112 carrying a stent 40 may then be introduced into the aorta 1, i.e., over the guidewire 98 and/or through the guide catheter 160. A distal end 116 of the delivery catheter 112 may be introduced over the guidewire 98 at least partially into the renal artery 4.

In one embodiment, the distal end 116 including the stent 40 may be advanced entirely through the opening 198 and into the branch 4. The distal end 116 may then be withdrawn to position the stent 40 adjacent the opening 198 and branch 4, e.g., such that a first portion 40a is disposed within the stent-graft 190 and a second portion 40b is disposed through the opening 198 and within the branch 4, as shown in FIG. 9A.

Turning to FIGS. 9A-10, the stent 40 on the delivery catheter 112 may be expanded and/or flared to deform and/or trap the graft material surrounding the opening 198 of the stent-graft 190 between the stent 40 and the vessel wall. For example, as shown in FIG. 9A, with the stent 40 partially disposed within the aorta 1, a proximal balloon 122a may be inflated to expand the first portion 40a of the stent 40, i.e., to flare the first portion 40a. Then, as shown in FIG. 9B, the delivery catheter 112 may be advanced until the flared first portion 40a contacts the stent-graft 190. For example, the delivery catheter 112 may be advanced with sufficient force to engage the flared first portion 40a within the opening 198 and/or press the stent-graft 190 against the vessel wall, e.g., as shown in FIG. 9B. As best seen in FIG. 9C, once fully deployed, the stent 40 has a flared shape that mechanically holds the material of the stent-graft 190 around the opening 198 against the vessel wall.

Turning to FIG. 10, a distal balloon 122b on the delivery catheter 112 may be expanded to expand a second or main portion 40b of the stent 40 within the renal artery 4, e.g., to dilate and/or substantially secure the stent 40 relative to the renal artery 4. Optionally, as shown in FIG. 10, a distal force may be applied to the delivery catheter 112 while the distal balloon 122b is expanded to substantially lock the stent 40 and stent-graft 190 relative to the aorta 1 and renal artery 4. If desired, additional inflation of the balloon(s) 122 may follow, e.g., to further flare the first portion 40a or otherwise expand and/or secure the stent 40, as disclosed in the applications identified above. Once fully deployed, the stent 40 may provide a substantially smooth transition and accurate alignment of the stent graft material to the ostium of the renal artery 4.

The balloons 122 may then be deflated, and the delivery catheter 112 may be withdrawn from the stent-graft 190 and aorta 1, e.g., through the guide catheter 160. Additional information on apparatus and methods for delivering the stent 40 into the aorta 1, i.e., through the opening 198, are disclosed in the applications identified above. If one or more additional stents 40 are to be delivered, e.g., into the renal artery 4 opposite that shown as being stented in FIGS. 9A-10, another delivery catheter (not shown), similar to delivery catheter 112 may be introduced, e.g., using a similar procedure to that described above. Once the desired number of stents 40 are delivered, the guide catheter 160 may be removed.

Turning to FIGS. 11A and 11B, a stent 40 is shown implanted through an opening 198 in a stent-graft 190, e.g., such as those described elsewhere herein. The stent 40 may secure the stent-graft 190 such that the stent-graft cannot migrate axially along the aorta 1, as shown in FIG. 11B where no stent 40 is provided. Such migration may risk separation of the stent-graft 190 from the aorta 1, which may result in a leak into the aneurysm 3. In addition, migration may partially obstruct flow into the renal artery 4, which may risk subsequent injury to the patient. The stent 40 may also provide a smooth transition, which may enhance flow from the aorta 1 into the renal artery 4 and/or reduce the risk of thrombosis or other recurrence of a lesion if one existed at the ostium of the renal artery 4.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for treating an aneurysm within a main body lumen that communicates with a branch body lumen, comprising:
a stent-graft comprising a tubular body for implantation within a main body lumen across an aneurysm, the tubular body comprising a first opening therethrough that may be aligned with the branch body lumen when the stent-graft is implanted across an aneurysm; and
a first stent comprising a first portion and a second portion, the second portion being receivable through the first opening in the tubular body, the first portion being expandable to a flared condition for engaging the tubular body around the first opening.

2. The apparatus of claim 1, further comprising a delivery device comprising a proximal end, a distal end sized for introduction into a body lumen, the first stent being carried on the distal end for delivering the stent into the stent-graft.

3. The apparatus of claim 2, wherein the delivery device comprises at least one expandable member on the distal end, at least a portion of the first stent being disposed on the distal end over the at least one expandable member.

4. The apparatus of claim 1, wherein the stent-graft comprises a second opening, the apparatus comprising a second stent comprising a first portion and a second portion, the second portion being receivable through the second opening in the tubular body, the first portion being expandable to a flared condition for engaging the tubular body around the second opening.

5. The apparatus of claim 4, wherein the second opening is disposed generally opposite the first opening.

6. A method for securing a stent-graft deployed within a main body lumen relative to a branch body lumen communicating with the main body lumen, the method comprising:
introducing a distal end of a delivery device into the main body lumen, the distal end carrying a first stent thereon;
advancing the distal end through a first opening in the stent-graft at least partially into the branch body lumen; and
expanding the first stent to anchor the stent-graft relative to the branch body lumen.

7. The method of claim 6, wherein the first stent is expanded such that a first portion of the first stent is flared to engage the stent-graft around the first opening.

8. The method of claim 7, wherein the first portion of the first stent is flared to provide a substantially smooth transition between the stent-graft and the branch body lumen.

9. The method of claim 7, wherein the first stent is expanded such that a second portion of the first is expanded within the branch body lumen, thereby securing the first stent relative to the branch body lumen.

10. The method of claim 6, wherein a distal force is applied to the delivery device while at least a portion of the first stent is expanded to press the stent-graft around the first opening against an ostium communicating between the main body lumen and the branch body lumen.

11. The method of claim 6, further comprising:
introducing a distal end of a delivery device into the main body lumen, the distal end carrying a second stent thereon;
advancing the distal end through a second opening in the stent-graft at least partially into the branch body lumen; and
expanding the second stent to anchor the stent-graft relative to the branch body lumen.

12. The method of claim 11, wherein the second opening is disposed generally opposite the first opening.

13. A stent comprising a tubular member including first and second ends defining a longitudinal axis therebetween and a plurality of cells disposed between the first and second ends, the tubular member being expandable from a contracted condition to an enlarged condition, the stent comprising:
a first set of cells disposed at the first end;
a second set of cells disposed adjacent the first set of cells; and
a plurality of connectors coupling the first set of cells with the second set of cells such that radial expansion of the second set of cells towards the enlarged condition causes the first set of cells to flare radially outwardly.

14. The stent of claim 13, wherein the first end has a larger cross-section than the second end in the enlarged condition.

15. The stent of claim 13, wherein the connectors comprise a plurality of generally axial struts coupling the first set of cells to the second set of cells.

16. The stent of claim 15, wherein the first and second cells comprise zigzag patterns including peaks and valleys, and wherein the struts connect respective peaks and valleys of the first and second sets of cells.

17. The stent of claim 16, wherein the struts coupling respective peaks of the first and second sets of cells are longer than struts coupling respective valleys of the first and second sets of cells.

18. The stent of claim 16, wherein the zigzag patterns include generally axial elements connecting the alternating peaks and valleys, and wherein the axial elements in the first set of cells are longer than the axial elements in the second set of cells.

19. An apparatus for delivering a stent into an ostium, comprising:
an elongate member comprising a proximal end, a distal end sized for introduction into a body lumen, and an expandable member on the distal end; and
a stent on the distal end, the stent comprising a first flaring portion and a second main portion, the second main portion overlying the expandable member such that the expansion of the expandable member causes the second main portion to expand radially,
the first flaring portion comprising a first band of cells adjacent a first end of the stent and a second band of cells between the first end and the second main portion, the first and second bands of cells connected by substantially axial connectors such that radial expansion of the second band of cells causes the first band of cells to flare radially outwardly.

20. The apparatus of claim 19, the second set of cells being coupled to the second main portion such that radial expansion of the second main portion causes the second set of cells to radially expand.
